(19) United States
(12) Patent Application Publication
(10) Pub. No.: US 2023/0080090 A1
(43) Pub. Date: Mar. 16, 2023

(54) SELECTIVE PURIFICATION OF RNA

(71) Applicant: ABBOTT MOLECULAR INC., Des Plaines, IL (US)

(72) Inventor: Gerard J. Gundling, Des Plaines, IL (US)

(21) Appl. No.: 17/898,140

(22) Filed: Aug. 29, 2022

Related U.S. Application Data
(60) Provisional application No. 63/238,346, filed on Aug. 30, 2021.

(51) Int. Cl. C12Q 1/6806; B82Y 5/00; B82Y 15/00

(52) U.S. Cl. CPC C12Q 1/6806; B82Y 5/00; B82Y 15/00

(57) ABSTRACT

The invention, in some aspects, pertains to compositions and methods for selective extraction and purification of RNA.

This application claims priority to U.S. provisional patent application Ser. No. 63/238,346, filed Aug. 30, 2021, which is incorporated herein by reference in its entirety

### FIELD OF THE INVENTION

The invention relates, in part, to novel compositions and methods for preferential extraction and purification of RNA.

### BACKGROUND

Conventional methods of nucleic acid purification typically use the binding and release of RNA and DNA from silica surfaces under non-selective conditions. These surfaces can be of many forms such as filters, immobilized particles, and magnetic particles coated with various form of silica. Such conventional methods are primarily used to isolate all types of nucleic acids in a sample. If it is necessary to have either purified RNA without purified DNA or purified DNA without purified RNA, a typical protocol would require digestion of the undesired nucleic acid with an appropriate nuclease followed by re-purification of the target nucleic acid type. For example, to isolate RNA without DNA, a typical protocol would require isolation of the total nucleic acids, digestion of the DNA with DNase, and then removal of the DNA breakdown products with a second purification of the RNA. Such conventional methods are not optimal for RNA recovery for sensitive assays, including but not limited to HIV analysis. Without selectivity at the extraction or purification stages, conventional methods do not maximize target RNA recovery, nor do they select against DNA recovery to minimize the presence of DNA to further diminish the possibility of interference with assay results.

Dried blood spots (DBS) are an important type of sample carrier, including but not limited to use as an established technique for analysis of HIV in blood samples (Cassol et al., J. Clin. Microbiol. (1991) 29(4), 667-671; Cassol et al., J. Clin. Microbiol. (1992) 30(12), 3039-3042; Nyambi et al., J. Clin. Microbiol. (1994) 32(11), 2858-2860). Blood samples are spotted on filter discs, dried, and stored. This process stabilizes the nucleic acids in the sample and allows them to be extracted at a later date. A DBS sample is incubated in a fluid to remove the nucleic acid target from the paper and the nucleic acid target is then processed for amplification and detection. Nucleic acid target processing may include the use of magnetic silica or iron oxide particles to bind the nucleic acids (U.S. Pat. No. 10,280,473, the disclosure of which is incorporated herein by reference). An alternative process has been described for the purification of HIV from DBS samples (U.S. Pat. No. 10,125,402, the disclosure of which is incorporated herein by reference). That process rehydrates a DBS with phosphate buffered saline, separating the cell-free viruses from any cell debris that may be present in the rehydrated dried blood sample by way of a filter, and measuring cell-free virus particles by a viral particle quantification technique. However, that process is not selective for RNA and claims that both DNA and RNA viruses can be isolated with that method, nor is there any disclosure of nucleic acid selection at the purification stage.

Thus there remains a need for a method that maximizes target RNA recovery for sensitive assays while minimizing DNA recovery to reduce the possibility of assay interference from background DNA.

### SUMMARY

According to an aspect of the disclosure, a two-step method of preferentially extracting RNA molecules from a sample dried on a solid carrier is provided, the method including: (a) providing a liquid biological sample dried on a solid carrier, wherein the liquid biological sample includes nucleic acids including RNA molecules; (b) providing an extraction buffer comprising less than 3.5 M GITC; (c) contacting the solid carrier with the extraction buffer, thereby preferentially releasing RNA molecules from the solid carrier into the extraction buffer; (d) isolating the extraction buffer of step (c) containing released RNA molecules; (e) suspending a plurality of copper-titanium oxide-coated (CuTi) magnetic particles in the isolated extraction buffer and incubating under conditions appropriate for binding of the released RNA molecules by the plurality of suspended CuTi particles; (f) capturing the plurality of CuTi particles and bound RNA molecules by application of a magnetic field; (g) removing the extraction buffer; and (h) contacting the plurality of CuTi particles and bound RNA molecules with an elution buffer, under conditions appropriate for release of the bound RNA molecules into the elution buffer. In some aspects, the liquid biological sample is whole blood. In some aspects, the liquid biological sample dried on a solid carrier is a dried blood spot (DBS). In certain aspects, the liquid biological sample dried on a solid carrier is suspected of containing a virus. In some aspects, the virus is human immunodeficiency virus 1 (HIV-1). In certain aspects, the virus is human papilloma virus (HPV). In some aspects, the extraction buffer further includes greater than 5% Tween®-20 and has a pH less than 6.0. In some aspects, the extraction buffer includes 3.2 M GITC, 7.5% Tween®-20, and has a pH of 5.6. In some aspects, the extraction buffer includes less than 3.2 M GITC, 7.5% Tween®-20, and has a pH less than 6.0. In certain aspects, step (e) further includes drawing the sequestered plurality of CuTi particles through an aqueous gel by means of a magnetic force, and step (g) is not performed. In certain aspects, the sequestered plurality of CuTi particles are drawn through the aqueous gel directly into the elution buffer of step (h). In some aspects, the elution buffer includes a low ionic strength buffer. In certain aspects, the elution buffer is water. In some aspects, the plurality of CuTi particles is present in a molar excess relative to the plurality of RNA molecules in the sample. In some aspects, the method is automated. In some aspects, the solid carrier includes filter paper. In some aspects, the method further includes (i) diagnosing a viral infection in a subject, wherein the diagnosing includes (1) obtaining a nucleotide sequence of the released RNA molecules, or of a template-directed polymerization product thereof, and (2) comparing the obtained nucleotide sequence of the released RNA molecules, or the template-directed polymerization product thereof, with a specific nucleotide sequence known to be present in virally infected cells, wherein a match between the compared nucleotide sequences is diagnostic of the viral infection in the subject. In certain aspects, the viral infection is an HIV infection.

In some embodiments, the present invention provides a method of extracting RNA molecules from a sample dried on a solid carrier, the method comprising: (a) providing a liquid biological sample dried on a solid carrier, wherein the liquid biological sample comprises nucleic acids including RNA molecules; (b) providing an extraction buffer comprising less than 3.5 M GITC; (c) contacting the solid carrier with the extraction buffer, thereby releasing RNA molecules from the solid carrier into the extraction buffer; (d) isolating the extraction buffer of step (c) containing released RNA molecules; (e) suspending a plurality of copper-titanium oxide-coated (CuTi) magnetic particles in the isolated extraction buffer and incubating under conditions appropriate for binding of the released RNA molecules by the plurality of suspended CuTi particles; (f) capturing the plurality of CuTi particles and bound RNA molecules by application of a magnetic field; (g) removing the extraction buffer; and (h) contacting the plurality of CuTi particles and bound RNA molecules with an elution buffer, under conditions appropriate for release of the bound RNA molecules into the elution buffer. In some embodiments, the liquid biological sample is whole blood. In some embodiments, the liquid biological sample dried on a solid carrier is a dried blood spot (DBS). In some embodiments, the liquid biological sample dried on a solid carrier is suspected of containing a virus. In some embodiments, the virus is human immunodeficiency virus 1 (HIV-1). In some embodiments, the virus is human papilloma virus (HPV). In some embodiments, the extraction buffer further comprises greater than 5% Tween®-20 and has a pH less than 6.0. In some embodiments, the extraction buffer comprises 3.2 M GITC, 7.5% Tween®-20, and has a pH of 5.6. In some embodiments, the extraction buffer comprises less than 3.2 M GITC, 7.5% Tween®-20, and has a pH less than 6.0. In some embodiments, step (e) further comprises drawing the sequestered plurality of CuTi particles through an aqueous gel by means of a magnetic force, and step (g) is not performed. In some embodiments, the sequestered plurality of CuTi particles are drawn through the aqueous gel directly into the elution buffer of step (h). In some embodiments, the elution buffer comprises a low ionic strength buffer. In some embodiments, the elution buffer is water. In some embodiments, the plurality of CuTi particles is present in a molar excess relative to the plurality of RNA molecules in the sample. In some embodiments, the method is automated. In some embodiments, the solid carrier comprises filter paper. In some embodiments, the method further comprises (i) diagnosing a viral infection in a subject, wherein the diagnosing comprises: (1) obtaining a nucleotide sequence of the released RNA molecules, or of a template-directed polymerization product thereof; and (2) comparing the obtained nucleotide sequence of the released RNA molecules, or the template-directed polymerization product thereof, with a specific nucleotide sequence known to be present in virally infected cells, wherein a match between the compared nucleotide sequences is diagnostic of the viral infection in the subject. In some embodiments, the viral infection is an HIV infection.

### BRIEF DESCRIPTION OF THE DRAWINGS

FIG. 1A-1C presents graphs illustrating the results of HIV RNA and cellular DNA recovery from DBS samples using silica particle purification. “AD,” “DB,” and “LB” indicate samples extracted with AD, DB, and LB buffers, respectively. FIG. 1A shows percentages of HIV RNA “(RNA)” and DNA “(DNA)” recovered from DBS samples using silica particles. FIG. 1B compares the ratios of HIV RNA copies per ng cellular DNA recovered from DBS samples “(DBS)” with silica particle purification to the ratios of HIV RNA copies per ng cellular DNA recovered from whole blood “(WB)” samples with silica particle purification. FIG. 1C shows the relative increases in RNA selectivity for each buffer for DBS samples versus whole blood samples (HIV RNA/cellular DNA ratio for DBS extraction with silica particle purification divided by HIV RNA/cellular DNA ratio for whole blood extraction with silica particle purification).

FIG. 2A-2C presents graphs illustrating the results of HIV RNA and cellular DNA recovery from whole blood samples using CuTi particle purification. “AD,” “DB,” and “LB” indicate samples extracted with AD, DB, and LB buffers, respectively. FIG. 2A shows percentages of HIV RNA “(RNA)” and DNA “(DNA)” recovered from whole blood samples using CuTi particles. FIG. 2B compares the ratios of HIV RNA copies per ng cellular DNA recovered from whole blood samples with CuTi particle purification “(CuTi, WB)” to the ratios of HIV RNA copies per ng cellular DNA recovered from whole blood samples with silica particle purification “(sil, WB).” FIG. 2C shows the relative increases in RNA selectivity for each buffer for CuTi particle purification versus silica particle purification (whole blood samples) (HIV RNA/cellular DNA ratio for CuTi particle purification divided by HIV RNA/cellular DNA ratio for silica particle purification).

FIG. 3A-3C presents graphs illustrating the results of HIV RNA and cellular DNA recovery from DBS samples using CuTi particle purification. “AD,” “DB,” and “LB” indicate samples extracted with AD, DB, and LB buffers, respectively. FIG. 3A shows percentages of HIV RNA “(RNA)” and DNA “(DNA)” recovered from DBS samples using CuTi particles. FIG. 3B compares the ratios of HIV RNA copies per ng cellular DNA recovered from DBS samples with CuTi particle purification “(CuTi, DBS)” to the ratios of HIV RNA copies per ng cellular DNA recovered from whole blood samples with silica particle purification “(sil, WB).” FIG. 3C shows the relative increases in RNA selectivity for each buffer for CuTi particle purification versus silica particle purification (DBS samples) (HIV RNA/cellular DNA ratio for CuTi particle purification divided by HIV RNA/cellular DNA ratio for silica particle purification)

FIG. 4 presents a graph illustrating the increased selectivity of RNA extraction and purification from DBS samples using LB, AD and DB buffers in combination with CuTi particle purification versus silica particle purification. Silica particle purification, circles; CuTi particle purification, diamonds. “AD”, AD buffer; “DB”, DB buffer; “LB”, LB buffer.

FIG. 5 presents a graph showing a bivariate fit of HIV mass ratio (MR) values and DNA content as a measure of assay performance for combinations of sample types, buffers tested, and particle purification used. The results are from DBS samples (filled symbols) and whole blood spiked samples (open symbols) using AD, DB, and LB buffers in combination with CuTi particle purification versus silica particle purification. Silica particle purification, circles; AD buffer=gray, no marker, DB buffer=“X” marker, LB buffer=“L” marker. CuTi particle purification, diamonds; AD buffer=gray, no marker, DB buffer=“X” marker, LB buffer=“L” marker. The solid line shows transformed fit to log.

### DETAILED DESCRIPTION

The present disclosure provides novel two-step methods for preferentially extracting RNA molecules from a sample dried on a solid carrier. Aspects of the disclosure are based, in part, on compositions and methods comprising simple reagents that can be easily assembled but embody a sophisticated design in order to address the problem of selectively enriching for RNA recovery from a sample. During a first step of methods of the disclosure, a selective extraction step, RNA is selectively released from a sample dried on a solid carrier when the solid carrier is contacted with an extraction buffer, thereby preferentially releasing RNA molecules from the solid carrier into the extraction buffer. The extraction buffer containing released RNA molecules is isolated and directly used for a second step of methods of the disclosure, a selective purification step. During the second step, the released RNA molecules are selectively purified when a plurality of copper-titanium oxide-coated (CuTi) magnetic particles (U.S. Pat. No. 10,392,613, the disclosure of which is incorporated herein by reference) is suspended in the isolated extraction buffer and incubated under conditions appropriate for binding of the released RNA molecules by the plurality of suspended CuTi particles. Bound RNA molecules are then released by capturing the plurality of CuTi particles by application of a magnetic field, removing the extraction buffer, and contacting the plurality of CuTi particles and bound RNA molecules with an elution buffer to remove contaminants, under conditions appropriate for release of the bound RNA molecules into the elution buffer. The resulting eluate is enriched for RNA because each stage preferentially increases the proportion of RNA relative to DNA. Thus, in embodiments of methods of the disclosure, both steps preferentially select for RNA, and the steps combined provide more powerful selection for RNA than does either step alone. Purified RNA can then be used in molecular assays with minimal DNA contamination that could confound or obscure results.

In some aspects, compositions and methods of the disclosure combine extraction and purification steps such that they are performed simultaneously or in a fashion that reduces or eliminates traditional washes and mechanical steps. In some embodiments, methods of the disclosure are automated. In some embodiments, methods of the disclosure are performed in an automated analytical instrument, such as the Abbott Alinity m (Abbott, Abbott Park, Ill.).

Aspects of compositions and methods of the disclosure build upon an earlier concept for nucleic acid extraction (U.S. Pat. No. 10,526,596, the disclosure of which is incorporated herein by reference), in which CuTi particles were used to bind nucleic acids. However, that system as described did not preferentially select for RNA extraction from a sample prior to using CuTi particles, and did not perform CuTi particle purification with a buffer and conditions optimized to enhance selectivity for RNA. An advantage of methods of the instant disclosure is that the combined use of a single extraction/purification reagent and CuTi particles selectively extracts RNA from a sample and greatly decreases the amount of cellular DNA present. Thus, methods of the disclosure eliminate the need to use an enzymatic treatment, such as DNase, to reduce cellular DNA levels prior to an assay. Preferentially increasing the proportion of RNA to DNA with each step of methods of the disclosure allows a more accurate determination of RNA levels by reducing amplification of cellular HIV DNA in the assay. In the context of HIV, for example, preferential selection for RNA over cellular DNA is important because cellular DNA may contain proviral DNA that can have an effect on target quantitation and can also have a negative impact on the performance of the assay. HIV proviral DNA integrates into cellular DNA and assays that use PCR amplification can amplify this proviral DNA resulting in over-quantification of HIV viral load (Wan, et al., (2010) J. Clin. Microbiol. 48(6) 2186-2190). Compositions and methods of the disclosure represent a substantial improvement to existing RNA extraction processes, and may be important for point-of-care testing and high-throughput processing of samples, for purposes including but not limited to medical diagnostics, detection of a viral infection, blood banking, and transplantation.

In aspects, the present disclosure relates to compositions and methods for preferentially extracting and purifying RNA from a biological sample. As used herein, the term “biological sample” refers to samples obtained from a subject, from cells, tissues, or other biological sources. Biological samples may be naturally occurring, may be concentrates or suspensions of cells or tissues or fragments thereof in a buffer, may be products of cells or tissues, or may be synthetic nucleic acids. Non-limiting examples of biological samples include blood, bone marrow, tissue, surgical specimen, biopsy specimen, liquid biopsy specimen, tissue explant, organ culture, or any other tissue or cell preparation, or fraction or derivative thereof or isolated therefrom, etc.

In aspects of the present disclosure, the biological sample is a liquid biological sample. Non-limiting examples of liquid biological samples include whole blood, serum, plasma, lymph, vitreous humor, aqueous humor, mucous, cerebrospinal fluid, saliva, urine, milk, ascites fluid, synovial fluid, peritoneal fluid, amniotic fluid, fermentation broths, cell culture products, nucleic acid synthesis products, or other biological fluid, etc. In aspects of the disclosure, the sample contains or is suspected of containing a virus. In some embodiments, the virus is an RNA virus, meaning that the viral genome is encoded by RNA. In some embodiments, the virus is a retrovirus, including but not limited to human immunodeficiency virus (HIV). In embodiments of the present disclosure, nucleic acids may be obtained from any biological sample including, for example, primary cells, cell lines, freshly isolated cells or tissues, frozen cells or tissues, paraffin-embedded cells or tissues, fixed cells or tissues, and/or laser dissected cells or tissues. In some embodiments, a sample from which nucleic acids are isolated for use in methods of the invention is a control sample. Nucleic acids may be isolated from a subject, cell, or other source according to methods known in the art. Persons having skill in the art will understand how to obtain and prepare biological samples and liquid biological samples using art-known methods, including but not limited to obtaining whole blood, preparing plasma from blood, isolating cells from biological fluids, homogenizing tissue, disrupting cells or viral particles, preparing liquids from solid materials, diluting viscous fluids, filtering liquids, distilling liquids, concentrating liquids, inactivating interfering components, adding reagents, purifying nucleic acids, and the like.

In aspects of the present disclosure, the liquid biological sample is applied to a solid carrier and the solid carrier is subsequently dried. In embodiments, the solid carrier is filter paper. In another embodiment, the solid carrier is a soluble fiber, including but not limited to soluble cellulose that dissolves when contacted with the extraction buffer. In a preferred embodiment, the liquid biological sample dried on a solid carrier is a dried blood spot (DBS). DBSs are advantageous for collecting and storing blood samples because are they easy to collect: only a finger prick or heal prick is necessary, bypassing the need for venipuncture. No phlebotomy skills are needed and collection equipment is minimal. Sample cards usually have an indication of spot size (diameter) to ensure adequate sample volume (usually a sample volume of about 70 μl is adequate). Samples may be air dried at ambient conditions. DBS do not require refrigerated storage and are stable for long periods of time at ambient conditions (weeks to months), and can be easily stored or transported in a closed container (such as Tupperware® or a sealed envelope). Thus, patient samples can be reliably collected at sites that may not also have medical facilities that can process standard patient samples and can be transported for testing without fear of loss of sample integrity. Because of the lower biohazard afforded with DBS samples, properly packaged samples can be mailed to a test facility (Shipping Guidelines for Dried-Blood Spot Specimens, CDC, cdc.gov/labstandards/pdf/nsqap/Bloodspot_Transportation_Guidelines.pdf, and references contained therein; Clinical Laboratory and Standards Institute. Blood collection on filter paper for newborn screening programs; Approved standard-Fifth edition. CLSI document LA4-A6. Wayne, Pa.: Clinical and Laboratory Standards Institute; 2012).

The terms “nucleic acid” or “nucleic acids” as used herein refer to a polymer comprising multiple nucleotide monomers. The term “nucleotide” as used herein includes a phosphoric ester of nucleoside—the basic structural unit of nucleic acids (DNA or RNA). A nucleic acid may be either single stranded, or double stranded with each strand having a 5′ end and a 3′ end. A nucleic acid may be RNA (including but not limited to viral RNA and non-viral RNA), DNA (including but not limited to cellular DNA, proviral DNA, cDNA, or genomic DNA), or hybrid polymers (e.g., DNA/RNA). The terms “nucleic acid” and “nucleic acids” do not refer to any particular length of polymer. Nucleic acids used in embodiments of compositions and methods of the disclosure may be at least 1, 2, 3, 4, 5, 10, 20, 30, 40, 50, 60, 70, 80, 90, 100, 200, 500, 1000, or 2000 kb, or more in length. The term “sequence,” used herein in reference to a nucleic acid, refers to a contiguous series of nucleotides that are joined by covalent bonds, such as phosphodiester bonds. A nucleic acid may be chemically or biochemically synthesized, or may be isolated from a subject, cell, tissue, or other biological sample or source that comprises, or is believed to comprise, nucleic acid sequences including, but not limited to RNA, mRNA, and DNA. Nucleic acids enriched, isolated, or purified using the composition of the present disclosure may be used in any conventional molecular assay or process known to those of ordinary skill in the art because the nucleic acids are not altered in any way that may be detrimental to their subsequent use. For example, the nucleic acids may be sequenced, amplified by PCR, used in expression vectors, etc. In this regard, the nucleic acids may be contacted with enzymes such as, for example, a DNA polymerase or a reverse transcriptase before or after elution, or before or after being pulled through an aqueous gel wash layer. Further, this disclosure contemplates that the nucleic acid may be sequenced on the plurality of CuTi particles without dilution. Further, this disclosure contemplates that the nucleic acids bound to the plurality of CuTi particles are contacted with bisulfite after passing through the gel wash layer and/or after elution such that unmethylated cytosines are deaminated. Further, this disclosure contemplates that at least one nucleic base in the nucleic acid has an epigenetic modification.

In aspects, compositions and methods of the disclosure involve an extraction buffer comprised of three components: a chaotropic agent that denatures proteins, disrupts cells and viruses, and helps prevent nucleic acid degradation by inactivating nucleases; a detergent; and a buffer to adjust pH. In some embodiments, the chaotropic agent is guanidine isothiocyanate (GITC). In some embodiments, the concentration of GITC in the extraction buffer is 3.5 M GITC, less than 3.5 M GITC, 3.2 M GITC, or less than 3.2 M GITC. In embodiments, the detergent is Tween®-20, a non-ionic detergent that also helps solubilize cellular material and assists in the purification process. In embodiments, the percentage of Tween®-20 in the extraction buffer is at least 5%, 6%, 7%, 7.5%, 8%, 9%, or 10%. In some embodiments, the buffer is Tris, or potassium (K) acetate. In embodiments, the pH of the extraction buffer is less than 6.0; in some embodiments, the pH is 5.6. In a preferred embodiment, the extraction buffer involves 3.5 M GITC, greater than 5% Tween®-20, and has a pH less than 6.0. In a preferred embodiment, the extraction buffer involves less than 3.5 M GITC, greater than 5% Tween®-20, and has a pH less than 6.0. In another preferred embodiment, the extraction buffer involves 3.2 M GITC, 7.5% Tween®-20, and has a pH of 5.6. In another preferred embodiment, the extraction buffer involves less than 3.2 M GITC, 7.5% Tween®-20, and has a pH less than 6.0.

In aspects of the disclosure, the solid carrier is contacted with the extraction buffer, thereby preferentially releasing RNA molecules from the sample dried on the solid carrier into the extraction buffer, after which the extraction buffer containing released RNA molecules is isolated. As used herein, “isolating” the extraction buffer refers to physically separating the extraction buffer containing released RNA molecules from the solid carrier. In some embodiments, isolating the extraction buffer involves removing the solid carrier but not the extraction buffer from the reaction vessel. In some embodiments, the extraction buffer is removed from the reaction vessel and transferred to another reaction vessel, for example by means of hand-pipetting or automated pipetting. In some embodiments, the isolated extraction buffer is stored prior to the purification step; one of skill in the art will understand that isolated extraction buffer would be stored under conditions sufficient to preserve the released RNA molecules from degradation.

In aspects of the disclosure, purifying the preferentially released RNA molecules from the isolated extraction buffer comprises the step of suspending a plurality of copper-titanium oxide-coated (CuTi) magnetic particles, as described elsewhere (U.S. Pat. No. 10,392,613, the disclosure of which is incorporated herein by reference) in the isolated extraction buffer and incubating the isolated extraction buffer and plurality of CuTi particles suspended therein under conditions appropriate for preferentially binding of the released RNA molecules by the plurality of suspended CuTi particles. In some embodiments, the plurality of magnetic CuTi particles is added to the extraction buffer with a minimum of fluid. In aspects of compositions and methods of the disclosure, the order in which the plurality of CuTi particles is added to the extraction buffer relative to the provision of the sample dried on a solid carrier may depend on multiple factors, including but not limited to the composition of the sample to be extracted, and whether the experiments to be performed will be performed on a benchtop or in an automated analysis instrument. In some embodiments, the plurality of CuTi particles is added to the extraction buffer prior to the addition of the sample. In some embodiments, the plurality of CuTi particles is added to the extraction buffer separately after addition of the sample. In preferred automated embodiments, the plurality of CuTi particles are on-board an automated analysis instrument in bulk and are added automatically.

The present disclosure is not limited to particular amounts of copper and titanium. In some embodiments, the CuTi is present at a ratio of approximately 2:1 Cu to Ti (e.g., 3:1, 2:1, 1:1, 1:2, 1:3, etc.). In some embodiments, the particles have a diameter of 0.5 to 50 μm (e.g., 0.5 μm, 1.0 μm, 1.5 μm, 2.0 μm, 5.0 μm, 10.0 μm, 20.0 μm, 30.0 μm, 40.0 μm, 50.0 μm, etc.). In some embodiments, particles and/or solid surfaces are comprised of organic polymers such as polystyrene and derivatives thereof, polyacrylates and polymethacrylates, and derivatives thereof or polyurethanes, nylon, polyethylene, polypropylene, polybutylene, and copolymers of these materials. In some embodiments, particles are polysaccharides, in particular hydrogels such as agarose, cellulose, dextran, Sephadex, Sephacryl, chitosan, inorganic materials such as e.g. glass or further metal oxides and metalloid oxides (in particular oxides of formula MeO, wherein Me is selected from, e.g., Al, Ti, Zr, Si, B, in particular Al₂O₃, TiO₂, silica and boron oxide) or metal surfaces, e.g. gold. In some embodiments, particles are magnetic (e.g., para-magnetic, ferrimagnetic, ferromagnetic or superparamagnetic). In some embodiments, the particles may have a planar, acicular, cuboidal, tubular, fibrous, columnar or amorphous shape, although other geometries are specifically contemplated. In some embodiments, particles are commercially available (e.g., obtained from ISK Magnetics, Valparaiso, IN; Qiagen, Venlo, The Netherlands; Promega Corporation, Madison, Wis.; Life Technologies, Carlsbad, Calif.; Ademtech, New York, N.Y.; and Sperotech, Lake Forest, Ill.). In some embodiments, the plurality of CuTi particles are present in a quantity calculated to represent a molar excess relative to the quantity of nucleic acids calculated to be present in a biological sample.

Once the released RNA molecules are bound to the plurality of CuTi particles, the plurality of CuTi particles and bound RNA may be attracted by positioning a magnet around or adjacent to the reaction vessel. The application of magnetic force draws the plurality of CuTi particles to the wall of the reaction vessel. A variety of magnet shapes may be used, including but not limited to a bar magnet, donut magnet, or electromagnet. The magnet may be handheld if the method is being manually performed on a benchtop, or may be a programmable component of an automated instrument. One of skill in the art will be able to select a magnet best suited for the intended application. In aspects, once the plurality of CuTi particles have been captured by being drawn to the reaction vessel wall by application of a magnetic field, the extraction buffer is removed. Next, the captured plurality of CuTi particles is contacted with an elution buffer to release bound RNA molecules from the CuTi particles under the appropriate conditions. One with skill in the art will understand that removing the extraction buffer and adding the elution buffer may be performed manually if the method is performed on a benchtop, or may be a programmable component of an automated instrument. One skilled in the art will further understand that some or all of the eluate comprising the isolated RNA molecules may be used for subsequent molecular assays, including but not limited to PCR, qPCR, and RT-PCR, or may be stored under appropriate conditions for future use.

An “elution buffer” according to the present disclosure may be any reagent or set of reagents that separates bound nucleic acid from the metal oxide of the CuTi particle. In some embodiments, the elution buffer is a low ionic strength elution buffer that uses a phosphate counter-ion to elute the nucleic acids. In some embodiments, the low ionic strength elution buffer is a phosphate buffer, for example, a 5 mM phosphate buffer. In some embodiments, the low ionic strength elution buffer comprises an organophosphate such as phosphoserine. In some embodiments, the low ionic strength elution buffer is an inorganic phosphate. In a preferred embodiment, the elution buffer is water. In some aspects, compositions and methods of the disclosure comprise an elution buffer as a layer adjacent to and in fluid communication with a gel wash layer.

Aspects of compositions and methods of the disclosure build upon an earlier concept for extraction (U.S. Pat. No. 9,803,230, the disclosure of which is incorporated herein by reference), in which an aqueous gel was used to remove lysis buffer contaminants from magnetic particles as they were magnetically drawn through the gel after capturing nucleic acids in a liquid lysis buffer. The movement through the gel “washed” the particles by removing GITC-containing buffer from the particles. Conventional silica preparation compositions and methods for binding and purifying nucleic acids rely on salting out nucleic acids onto silica surfaces, and require washes with high levels of ethanol or other alcohols to remove lysis buffer contaminants. Those alcohols must also be removed by drying before elution due to the inhibitory nature of the alcohols in PCR-based tests. In contrast to conventional silica compositions and methods, CuTi particles retain nucleic acids under very low ionic strength conditions, allowing them to be washed with water to remove contaminants without eluting bound nucleic acids. Furthermore, CuTi particles do not require alcohol for sample processing washes and do not require any drying steps prior to elution. Consequently, those properties allow CuTi particles to be washed to remove lysis contaminants by being magnetically drawn through a low ionic strength aqueous gel. In embodiments of the disclosure, moving the magnet drives the migration of attracted CuTi particles and bound RNA at least partially through the aqueous gel (or “gel wash layer”) thereby removing extraction contaminants. In some embodiments, further application of the magnetic force may be used to drive the migration of attracted CuTi particles and bound RNA into an elution buffer after the attracted CuTi particles and bound RNA have been drawn through the gel wash layer. In some embodiments of the disclosure, a gel wash layer is present within the reaction vessel and is in fluid communication with either or both of the extraction buffer and the elution buffer. In embodiments, the elution buffer may be in fluid communication with the gel wash layer, or may be within a separate container. As described above herein, the elution buffer may be a low ionic strength buffer, may further be a phosphate buffer, or may further be water. Some or all of the eluted nucleic acids may be manually transferred to an assay or may be robotically transferred as part of an automated assay performed by an automated molecular analysis instrument. As discussed above herein, the isolated nucleic acids may be used for subsequent molecular assays, including but not limited to PCR, qPCR, and RT-PCR. In some embodiments, some or all of the eluted nucleic acids may be stored for use in future applications. In some embodiments, direct amplification or sequencing of nucleic acids bound to the plurality of CuTi particles is contemplated, wherein after the plurality of CuTi particles and bound RNA have been drawn through the gel wash layer, further application of the magnetic force may be used to draw the CuTi particles and bound RNA directly into a molecular assay.

As used herein, “reaction vessel” (which may also be referred to elsewhere herein as a “tube”) refers to any container in which the extraction and/or purification steps of the two-step method of the disclosure are performed. The material of which the reaction vessel is made is not critical so long as the material in no way interferes with aspects of the disclosed method for extraction and isolation of nucleic acids from a biological sample. For example, as discussed elsewhere herein, a magnetic field is utilized for the purpose of attracting magnetic particles. Given the importance of the magnetic field, the use of magnetic metals should be avoided. This requirement does not preclude all metals as, for example, austenite stainless steel structures will not be magnetic. Stainless steel having a ferrite or martensite structure will be magnetic and should be avoided. Glass and polymer formulations (e.g., polystyrene and polyethylene) are preferred for use in the formation of a reaction vessel.

The reaction vessel may comprise a tube of substantially circular cross-section, having a top and a bottom. A substantially circular cross-section is preferred (but not required) based on typical stock availability and prevalence of such cross-sections among materials used and consumed in the chemical and life-sciences industries. Non-limiting examples of tubes that may be used include but are not limited to 5 ml test tubes, or reaction vessels of custom design for use in instruments. In some embodiments of the disclosure, the top and the bottom of the tube are reversibly sealed. In some embodiments, only the top or the bottom of the tube is reversibly sealed. Non-limiting examples of materials that may be used to seal the top and bottom of the tube include a meltable hydrophobic wax, a meltable polymerizable material, or a removable plastic tip. In some embodiments, the top and/or bottom of the tube may be irreversibly sealed by a puncturable seal. One of skill in the art will understand how to choose a type of seal appropriate for a particular set of working conditions.

In some embodiments, an aqueous gel wash layer of the disclosure may be manually layered within a reaction vessel. In other embodiments, a reaction vessel may be manufactured for disposable use with an automated molecular diagnostics analysis instrument. Reaction vessel size may vary in terms of volume, length, and configuration depending on how methods of the disclosure are to be performed (for example, but not limited to, in a manual benchtop format, with an automated analysis instrument, or a combination thereof) and initial sample volume. As a non-limiting example, a reaction vessel for manual benchtop use may have an overall volume of at least 1 ml, 2 ml, 5 ml, 10 ml, or more. A reaction vessel for use with an automated analytical instrument may have a smaller volume and/or length, for example but not limited to at least 0.25 ml, 0.5 ml, 1 ml, or more. In certain embodiments, the tube may be vertically oriented, such that the top and bottom openings are directly aligned.

While the disclosed methods may be practiced manually in a benchtop format, in instances, the reaction vessel will be selected for use specifically with an automated analytical instrument. For example, the Abbott Alinity m (Abbott, Abbott Park, Ill.) is a fully integrated and automated molecular diagnostics analysis instrument with application, for example to polymerase chain reaction assays.

Aspects of methods of the disclosure involve an additional step of diagnosing a viral infection in a subject. As used herein, a viral infection, which may also be referred to as a viral disease, results in a cell or subject when a pathogenic virus is present in a cell or subject, or contacts a cell or subject, and infectious virus particles (virions) attach to and enter one or more cells. A viral infection in a cell, as referenced herein, means a cell into which virions have entered. A virally infected cell may be in a subject (in vivo) or obtained from a subject. In some embodiments, a virally infected cell is a cell in culture (in vitro), or is an infected cell obtained from culture. Numerous viruses, including such as retroviruses (including but not limited to human immunodeficiency virus (HIV) and human T-cell lymphotropic virus types 1 and 2 (HTLV-1, HTLV-II)) and RNA viruses (including but not limited to Orthomyxoviruses, Hepatitis C Virus (HCV), Ebola, coronaviruses, SARS, SARS-CoV-2, influenza, polio, and measles) are known to infect subjects and cells.

In aspects of the disclosure, diagnosing a viral infection in a subject comprises (1) obtaining a nucleotide sequence of the released RNA molecules, or of a template-directed polymerization product thereof, and (2) comparing the obtained nucleotide sequence of the released RNA molecules, or the template-directed polymerization product thereof, with a specific nucleotide sequence known to be present in virally infected cells, wherein a match between the compared nucleotide sequences is diagnostic of the viral infection in the subject. One skilled in the art will recognize that the released RNA molecules comprise a heterogeneous population, and a nucleotide sequence obtained from the released RNA molecules may therefore comprise one or more nucleotide sequences that differ from each other by one or more nucleotides. Nucleotide sequences of the released RNA molecules may be obtained by means of directly sequencing the RNA molecules through art-known methods, or by producing and sequencing template-directed polymerization products of the RNA molecules according to art-known methods, for example though not intended to be limiting, by reverse transcription PCR (RT-PCR), quantitative RT-PCR (qRT-PCR), or real-time RT-PCR. One of skill in the art will understand how to compare sequences obtained from the released RNA molecules or template-directed polymerization products thereof to other known sequences, including viral sequences, using art-known bioinformatics methods. In embodiments, the specific nucleotide sequence known to be present in virally infected cells is a sequence from a virus. In embodiments, the disclosed compositions and methods can be practiced manually in a benchtop format, or within an automated analytical instrument. For example, though not intended to be limiting, the Abbott Alinity m (Abbott, Abbott Park, Ill.) is a fully integrated and automated molecular diagnostics analysis instrument with application, for example to polymerase chain reaction assays.

As used herein, the term “subject” may refer to human or non-human animals, including mammals and non-mammals, vertebrates and invertebrates, and may also refer to any multicellular organism or single-celled organism such as a eukaryotic (including plants and algae) or prokaryotic organism, archaeon, microorganisms (e.g., bacteria, archaea, fungi, protists, viruses), and aquatic plankton. A subject may be considered to be a normal subject or may be a subject known to have or suspected of having a disorder, disease, or condition. Non-limiting examples of diseases or conditions include infectious diseases, such as retroviruses (including but not limited to human immunodeficiency virus (HIV) and human T-cell lymphotropic virus types 1 and 2 (HTLV-1, HTLV-II)) and RNA viruses (including but not limited to Orthomyxoviruses, Hepatitis C Virus (HCV), Ebola, coronaviruses, SARS, SARS-CoV-2, influenza, polio, and measles); monogenic disorders, such as sickle cell anemia, hemophilia, cystic fibrosis, Tay Sachs disease, Huntington's disease, and fragile X syndrome; chromosomal disorders, such as Down syndrome and Turner syndrome; polygenic disorders such as Alzheimer's disease, heart disease, diabetes, etc.; structural disorders such as deletions, insertions, and repeat expansions; and cancers.

Cells, tissues, or other sources or samples may include a single cell, a variety of cells, or organelles. It will be understood that a cell sample comprises a plurality of cells. As used herein, the term “plurality” means more than one. In some instances, a plurality of cells is at least 1, 10, 100, 1,000, 10,000, 100,000, 500,000, 1,000,000, 5,000,000, or more cells. A plurality of cells from which nucleic acids are isolated for use in compositions and methods of the disclosure may be a population of cells. A plurality of cells may include cells that are of the same cell type. In some embodiments, a cell from which nucleic acids are isolated for use in methods of the disclosure is a healthy normal cell, which is not known to have a disease, disorder, or abnormal condition. In some embodiments, a plurality of cells from which nucleic acids are isolated for use in methods of the disclosure includes cells having a known or suspected disease or condition or other abnormality, for example, a cell obtained from a subject diagnosed as having a disorder, disease, or condition, including, but not limited to a cell infected with a virus, a degenerative cell, a neurological disease-bearing cell, a cell model of a disease or condition, an injured cell, etc. In some embodiments, a cell is an abnormal cell obtained from cell culture, a cell line known to include a disorder, disease, or condition, including the non-limiting examples of disorders, disease, or conditions described elsewhere herein. In some embodiments of the invention, a plurality of cells is a mixed population of cells, meaning all cells are not of the same cell type. In some embodiments, a cell from which nucleic acids are isolated for use in methods of the invention is a control cell.

### EXAMPLES

### Materials and Methods

The following materials and methods were used in Examples 1-3 as described therein.

### Blood Samples and Sample Preparation

The experiments described in Examples 1-3 used a single set of whole blood and DBS samples prepared with defective HIV virus. Whole blood (ProMedDx, Norton, Mass.) was mixed with the defective virus sample and was tested as both an intact blood sample and, after spotting, as a DBS sample. Specifically, blood samples containing defective HIV virus particles were produced as follows. Defective HIV virus (hereafter “HIV”) stock (1.89×E\hat{ }8 particles/ml) was diluted to 1.85×E\hat{ }5 particles/ml in negative plasma diluent (5 μl of HIV stock into 5 ml negative plasma diluent). The diluted HIV sample was then further diluted to 5,000 copies/ml in whole blood (264 μl diluted HIV sample into 10 ml whole blood). HIV blood samples were then gently mixed by rocking. HIV blood samples contained 125 copies of HIV per 25 μl. DBS samples were prepared by spotting blood spotting cards (Whatman 903 Perforated, Whatman, Marlborough, Mass.) with five 25 μl spots per card. Cards were dried overnight and stored at −70° C.

### Extraction Buffers and Extraction Protocol

The experiments described in Examples 1-3 used three extraction buffers, which differed in their respective concentrations of guanidine iso-thiocyanate (GITC) and Tween®-20, and also in their respective pHs. Potassium acetate was used to buffer the solutions of the two buffers with a pH below 7 (AD and DB buffers) and Tris was used to buffer the solution of the buffer with a pH above 7 (LB buffer). Buffer compositions were as follows:

AD buffer: 3.2 M GITC, 7.5% Tween®-20, 50 mM K acetate and pH 5.6;

DB buffer: 3.5 M GITC, 5% Tween®-20, 50 mM K acetate and pH 6.0; and

LB buffer: 4.7 M GITC, 10% Tween®-20, 100 mM Tris and pH 8.0.

### Extraction Protocol

Two paper discs with 25 μl blood spots were incubated in 1.0 ml of extraction buffer at 55° C. for 30 minutes without shaking. The incubated extractions were then shaken at 1000 RPM for 1 minute on a heated orbital shaker (Thermomixer, Eppendorf, Framingham, Mass.). 1 ml of the extracted samples were then used in the purification step.

Whole blood samples were not extracted, but 25 μl of whole blood samples were added to 1.0 ml of extraction buffer for use in the purification process.

### Purification Processes

Two purification processes were used. One was a silica particle process requiring ethanol in the protocol. The other was a CuTi particle process that did not use ethanol in the protocol.

### Silica Purification

Purification with silica particles in Examples 1-2 was performed as follows. Samples were processed in a 5 ml tube (5 ml reaction vessel (RV) Abbott, Abbott Park, Ill.). For each sample, 1 ml sample, 0.4 ml ethanol, 25 μl silica particles (mParticles, Abbott, Abbott Park, Ill.), and 17 μl internal control (IC, HIV assay kit, Abbott, Abbott Park, Ill.) were added to an RV. Samples were incubated in a 50° C. Thermomixer (Eppendorf, Framingham, Mass.) for 20 minutes, mixing at 1000 RPM. Particles were collected with a magnet and supernatant was removed. Thermomixer (Eppendorf, Framingham, Mass.) temperature was increased to 75° C. For wash 1, 800 μl lysis buffer (LB buffer) was added to each tube, and tubes were vortexed for several seconds. Particles were collected with a magnet and supernatant was removed. For washes 2 and 3, 800 μμl 70% ethanol (Sigma-Aldrich, St. Louis, Mo.) was added to each tube, and tubes were vortexed for several seconds. After each vortexing, particles were collected with a magnet and supernatant was removed. After wash 3, any residual fluid was removed with a pipette and extractions were dried at 65° C. for 5 minutes. 100 μl of an RNA elution buffer was added and tubes were vortexed for several seconds. Tubes were then incubated for 10 minutes in a 75° C. Thermomixer (Eppendorf, Framingham, Mass.) with mixing at 1000 RPM. Particles were collected with a magnet, and the supernatant was collected and transferred to a new tube (VWR 0.65 ml, VWR, Radnor, Pa.).

### CuTi Purification

Purification with CuTi particles in Examples 2-3 was performed as follows. CuTi particles are suspended in 50 mM NaOH, 22% NaCl. Samples were processed in a 5 ml tube (5 ml reaction vessel (RV) Abbott, Abbott Park, Ill.). For each sample, 1 ml sample, a volume of water (0.75 ml for AD buffer; 1.0 ml for DB buffer; or 1.5 ml for LB buffer), 25 μl of a 2.5% w/v suspension of CuTi particles, and 17 μl internal control (IC, HIV assay kit, Abbott, Abbott Park, Ill.) were added to an RV. Samples were incubated in a 50° C. Thermomixer (Eppendorf, Framingham, Mass.) for 20 minutes, mixing at 1000 RPM. Particles were collected with a magnet and supernatant was removed. Thermomixer (Eppendorf, Framingham, Mass.) temperature was increased to 75° C. For wash 1, 800 μl lysis buffer (LB buffer) was added to each tube, and tubes were vortexed for several seconds. Particles were collected with a magnet and supernatant was removed. For washes 2 and 3, 800 μl Alinity m Diluent Solution (Abbott, Abbott Park, Ill.) was added to each tube, and tubes were vortexed for several seconds. After each vortexing, particles were collected with a magnet and supernatant was removed. After wash 3, any residual fluid was removed with a pipette, 100 μl RNA elution buffer was added and the samples were vortexed for several seconds. Tubes were then incubated for 10 minutes in a 75° C. Thermomixer (Eppendorf, Framingham, Mass.) with mixing at 1000 RPM. Particles were collected with a magnet, and the supernatant was collected and transferred to a new tube (VWR 0.65 ml, VWR, Radnor, Pa.).

### Real-Time Reverse Transcriptase PCR (RT-PCR) Assays

### HIV Assay

271 μl activator and 941 μl oligomix were added into an enzyme vial, and the mix was pipetted. 50 μl of the mix and 50 μl of sample were added into an optical reaction plate well. The reaction plate was sealed and the 0.6 ml HIV-1 assay program version 7.0 was run in an Alinity m2000RT cycler (Abbott, Abbott Park, Ill.). Standard curves for the quantitation of HIV and genomic DNA were determined using samples with known concentrations of HIV and genomic DNA according to established methods. Briefly, 0.5 ml plasma samples containing defective HIV at 5000 copies/ml were extracted using the CuTi particle method described elsewhere herein, eluted with 200 μl elution buffer, and the eluates were pooled. The eluate contained 625 HIV copies per 50 μl eluate. The pooled eluates were then serially diluted, 1:1 with elution buffer to contain 312.5, 156, 78, 39, and 19.5 HIV copies per 50 μl. Those standard curve samples were then assayed using the HIV assay as described herein with a 50 μl input of the standards. The cycle threshold values (CT) were then used to construct standard curves using JMP software.

### HPV Human Beta-Globin DNA Internal Control Assay

The HPV assay detected human beta-globin DNA as an internal control. 278 μl of activator and 402 μl of oligomix were added into an enzyme vial and the mix was pipetted. 25 μl of mix and 25 μl of sample were added into optical reaction plate wells. The reaction plate was sealed and the 0.4 ml HR HPV assay version 2.0 program was run in an Alinity m2000RT cycler (Abbott, Abbott Park, Ill.).

### Example 1

### Baseline Purification and Extraction Selectivity

### Results and Discussion

### (1) Silica Purification of HIV RNA and Cellular DNA From Whole Blood Samples

To determine a baseline for purification selectivity of the three extraction buffers, whole blood samples were purified using the silica particle process and each of the three extraction buffers (AD, DB, and LB buffers). The purified samples were then assayed with an HIV assay and an HPV assay for β-globin as an internal control. Levels of HIV RNA and cellular DNA were determined from assay CT values and standard curves. The ratio of HIV copies to the amount of cellular DNA was then used to determine the baseline selectivity of the process.

The results shown in Tables 1-3 demonstrate that the three different buffers had an impact on the amount of HIV RNA and cellular DNA isolated from the whole blood samples using the silica particle process. The highest levels of isolated HIV RNA copies were obtained using the DB buffer, and the AD and the LB buffer isolated the same amount (46% of the amount obtained with the DB buffer) (Table 1). The highest levels of cellular DNA were obtained using the DB and AD buffers, with the LB buffer isolating 47% of the highest amount (Table 2). The ratio of the number of HIV copies per 25 μl sample to nanograms of cellular DNA per 25 μl sample (Table 3) showed that there may be a two-fold difference between the AD buffer and the DB and LB buffers. The ratio of HIV RNA levels to cellular DNA levels was used as a measurement of the selectivity of the processes as described elsewhere herein.

TABLE 1
HIV RNA copies per 25 μl sample from
whole blood with silica purification

| | Mean | Std | Std Err | Lower | Upper |
|---|---|---|---|---|---|
| Buffer | (# RNA copies) | Dev | Mean | 95% | 95% |
| AD buffer | 232.313 | 154.181 | 77.090 | −13.0 | 477.65 |
| DB buffer | 543.699 | 118.199 | 48.254 | 419.7 | 667.74 |
| LB buffer | 231.057 | 112.912 | 50.496 | 90.9 | 371.26 |

TABLE 2
Cellular DNA (ng) per 25 μl sample
from whole blood with silica purification

| | Mean | Std | Std Err | Lower | Upper |
|---|---|---|---|---|---|
| Buffer | (ng DNA) | Dev | Mean | 95% | 95% |
| AD buffer | 378.320 | 28.9800 | 14.490 | 332.21 | 424.43 |
| DB buffer | 395.441 | 81.6324 | 33.326 | 309.77 | 481.11 |
| LB buffer | 181.877 | 38.0202 | 17.003 | 134.67 | 229.09 |

TABLE 3
HIV RNA copies/ng cellular DNA per 25 μl
sample from whole blood with silica purification

| | Mean | | | | |
|---|---|---|---|---|---|
| | (#RNA copies/ | Std | Std Err | Lower | Upper |
| Buffer | ng DNA) | Dev | Mean | 95% | 95% |
| AD buffer | 0.62522 | 0.408367 | 0.20418 | −0.0246 | 1.2750 |
| DB buffer | 1.45708 | 0.558837 | 0.22814 | 0.8706 | 2.0435 |
| LB buffer | 1.35438 | 0.797782 | 0.35678 | 0.3638 | 2.3450 |

### (2) Extraction Selection: Silica Extraction of HIV RNA and Cellular DNA From Dried Blood Spots

DBS samples were purified using the silica particle process and the three extraction buffers. The purified samples were then assayed with the HIV and the HPV assays and the levels of HIV and DNA were determined from the assay CT values and the standard curves described above herein. Higher levels of isolated HIV RNA copies were obtained with the AD and DB buffers than with the LB buffer (Table 4). The highest levels of cellular DNA were obtained with the DB buffer (Table 5).

TABLE 4
HIV RNA copies per 25 μl sample from
DBS extraction with silica purification.

| | Mean | Std | Std Err | Lower | Upper |
|---|---|---|---|---|---|
| Buffer | (# RNA copies) | Dev | Mean | 95% | 95% |
| AD buffer | 222.139 | 47.7785 | 19.506 | 172.00 | 272.28 |
| DB buffer | 242.943 | 79.8960 | 32.617 | 159.10 | 326.79 |
| LB buffer | 132.604 | 24.0685 | 9.826 | 107.35 | 157.86 |

TABLE 5
Cellular DNA (ng) per 25 μl from
DBS extraction with silica purification.

| | Mean | Std | Std Err | Lower | Upper |
|---|---|---|---|---|---|
| Buffer | (ng DNA) | Dev | Mean | 95% | 95% |
| AD buffer | 44.951 | 9.9574 | 4.065 | 34.501 | 55.40 |
| DB buffer | 101.991 | 28.5933 | 11.673 | 71.984 | 132.00 |
| LB buffer | 52.293 | 12.9882 | 5.302 | 38.663 | 65.92 |

### (3) RNA Selectivity: Comparing DBS RNA and DNA Recovery With Whole Blood Extraction RNA and DNA Recovery

The amounts of HIV RNA and cellular DNA (ng) extracted from the DBS samples and purified with the silica process in part (2) were compared to the amounts purified from whole blood samples with the silica process in part (1). The ratio of HIV RNA copies to the amount of cellular DNA (ng) was then used to determine the selectivity of the extraction process using each of the three extraction buffers in comparison to the baseline silica particle purification process.

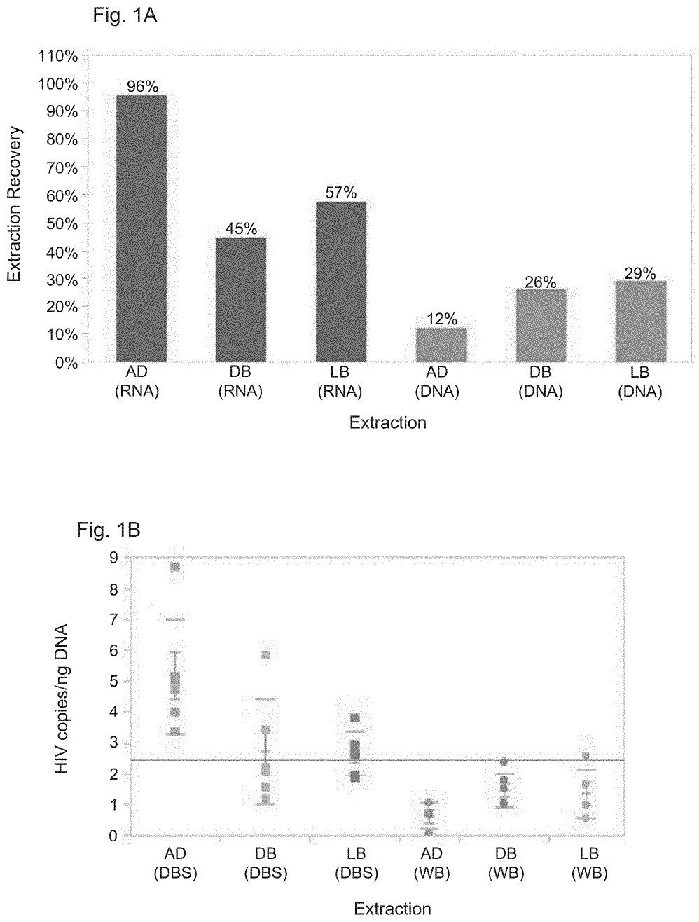

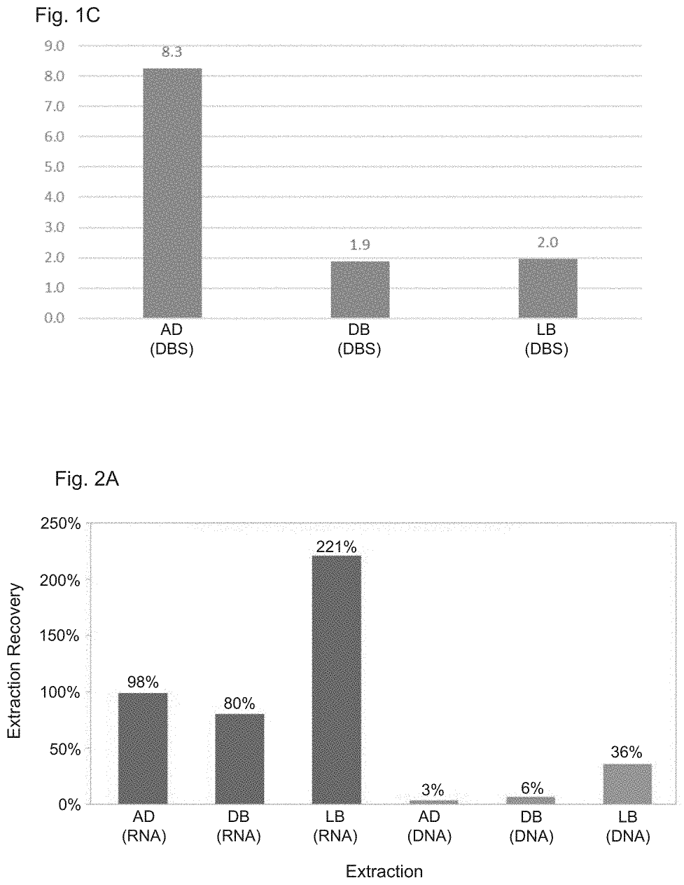

The data presented in this Example showed that HIV RNA recovery from DBS samples was greater than cellular DNA recovery from DBS samples compared to the respective quantities recovered from whole blood (FIG. 1A). The ratio of HIV RNA copies per 25 μl blood to the amount of cellular DNA (ng) per 25 μl blood increased from two-fold for the DB and LB buffers to over eight-fold for the AD buffer, showing that extraction from the DBS samples favored extraction of HIV RNA over that of cellular DNA (FIG. 1B, Table 6, and FIG. 1C). These results also indicated that extraction buffer composition influences the degree of selectivity at this stage.

TABLE 6
HIV RNA copies per ng cellular DNA from DBS extraction with
silica purification and silica purification from whole blood.

| | Mean | | | | |
|---|---|---|---|---|---|
| | (#RNA copies/ | Std | Std Err | Lower | Upper |
| Buffer | ng DNA) | Dev | Mean | 95% | 95% |

DBS extraction with silica purification

| | | | | | |
|---|---|---|---|---|---|
| AD buffer | 5.16769 | 1.85688 | 0.75807 | 3.219 | 7.1164 |
| DB buffer | 2.71955 | 1.71109 | 0.69855 | 0.924 | 4.5152 |
| LB buffer | 2.66274 | 0.71249 | 0.29087 | 1.915 | 3.4104 |

Silica purification from whole blood

| | | | | | |
|---|---|---|---|---|---|
| AD buffer | 0.62522 | 0.408367 | 0.20418 | −0.0246 | 1.2750 |
| DB buffer | 1.45708 | 0.558837 | 0.22814 | 0.8706 | 2.0435 |
| LB buffer | 1.35438 | 0.797782 | 0.35678 | 0.3638 | 2.3450 |

### Example 2

### Purification Selection

### Results and Discussion

(1) RNA Selectivity: HIV RNA and Cellular DNA Recovery From Whole Blood Extraction, CuTi Particles vs. Silica Process

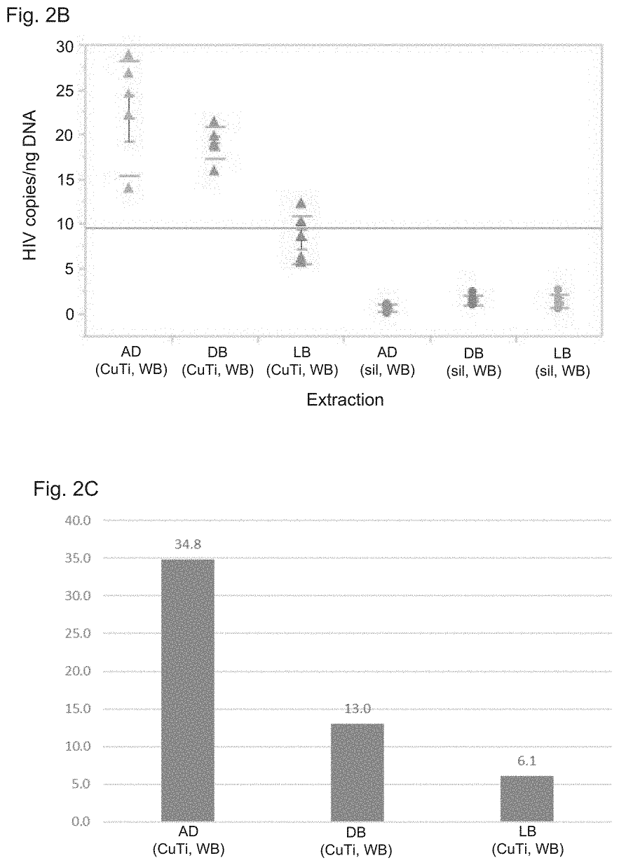

Whole blood samples were purified using the CuTi particle purification process and the three extraction buffers. Purified samples were then assayed with the HIV and HPV assays and the levels of HIV RNA (Table 7) and cellular DNA (Table 8) were determined from assay CT values and the standard curves described above herein. The ratio of HIV RNA copies to the amount of cellular DNA (ng) was then used to determine the selectivity of the CuTi particle purification process for HIV RNA, and to compare it to the selectivity of silica particle purification from whole blood samples. Whole blood silica purification data from Example 1 was used for comparison in Tables 7, 8, and 9, and FIG. 2A-2C.

TABLE 7
HIV RNA copies per 25 μl sample, comparing CuTi purification
from whole blood and silica purification from whole blood.

| | Mean | Std | Std Err | Lower | Upper |
|---|---|---|---|---|---|
| Buffer | (#RNA copies) | Dev | Mean | 95% | 95% |

HIV RNA extraction (whole blood) with CuTi purification

| | | | | | |
|---|---|---|---|---|---|
| AD buffer | 228.465 | 57.322 | 23.402 | 168.3 | 288.62 |
| DB buffer | 433.845 | 59.201 | 24.169 | 371.7 | 495.97 |
| LB buffer | 509.858 | 86.286 | 35.226 | 419.3 | 600.41 |

HIV RNA extraction (whole blood) with silica purification

| | | | | | |
|---|---|---|---|---|---|
| AD buffer | 232.313 | 154.181 | 77.090 | −13.0 | 477.65 |
| DB buffer | 543.699 | 118.199 | 48.254 | 419.7 | 667.74 |
| LB buffer | 231.057 | 112.912 | 50.496 | 90.9 | 371.26 |

TABLE 8
Cellular DNA (ng) per 25 μl sample, comparing CuTi purification
from whole blood and silica purification from whole blood.

| | Mean | Std | Std Err | Lower | Upper |
|---|---|---|---|---|---|
| Buffer | (ng DNA) | Dev | Mean | 95% | 95% |

Cellular DNA extraction (whole blood) with CuTi purification

| | | | | | |
|---|---|---|---|---|---|
| AD buffer | 10.630 | 0.6233 | 0.254 | 9.98 | 11.28 |
| DB buffer | 22.932 | 3.0016 | 1.225 | 19.78 | 26.08 |
| LB buffer | 64.897 | 12.9428 | 5.284 | 51.31 | 78.48 |

Cellular DNA extraction (whole blood) with silica purification

| | | | | | |
|---|---|---|---|---|---|
| AD buffer | 378.320 | 28.9800 | 14.490 | 332.21 | 424.43 |
| DB buffer | 395.441 | 81.6324 | 33.326 | 309.77 | 481.11 |
| LB buffer | 181.877 | 38.0202 | 17.003 | 134.67 | 229.09 |

A greater percentage of extracted HIV RNA than of extracted cellular DNA was recovered from whole blood using CuTi particles (FIG. 2A). HIV RNA recovery was comparable to the recovery with the silica process with DB and AD buffers, and increased with LB buffer (Table 7). However, the amount of cellular DNA recovered was much lower using the CuTi particle process (Table 8). The ratio of HIV RNA copies per 25 μl to the ng cellular DNA per 25 μl sample increased from six-fold for LB buffer, to 13-fold for DB buffer and to over 30-fold for AD buffer (Table 9, FIG. 2B and 2C).

TABLE 9
HIVcopies/ng cellular DNA CuTi vs Silica
purification from whole blood.

| | Mean | | | | |
|---|---|---|---|---|---|
| | (#RNA copies/ | Std | Std Err | Lower | Upper |
| Buffer | ng DNA) | Dev | Mean | 95% | 95% |

HIV RNA/cellular DNA recovery with CuTi purification (whole blood)

| | | | | | |
|---|---|---|---|---|---|
| AD buffer | 21.7757 | 6.41955 | 2.6208 | 15.04 | 28.513 |
| DB buffer | 18.9856 | 1.77169 | 0.7233 | 17.13 | 20.845 |
| LB buffer | 8.2227 | 2.66923 | 1.0897 | 5.42 | 11.024 |

HIV RNA/cellular DNA recovery with silica purification (whole blood)

| | | | | | |
|---|---|---|---|---|---|
| AD buffer | 0.6252 | 0.40837 | 0.2042 | −0.0246 | 1.275 |
| DB buffer | 1.4571 | 0.55884 | 0.2281 | 0.87 | 2.044 |
| LB buffer | 1.3544 | 0.79778 | 0.3568 | 0.36 | 2.345 |

### (2) RNA Selectivity, RNA and DNA Recovery From DBS Samples: CuTi Purification Selection

### Results and Discussion

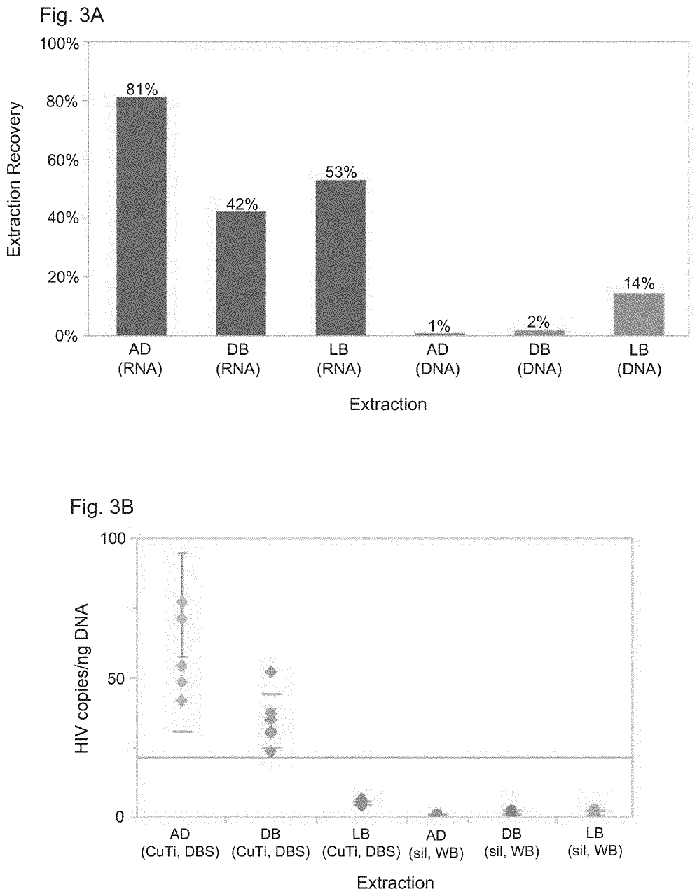

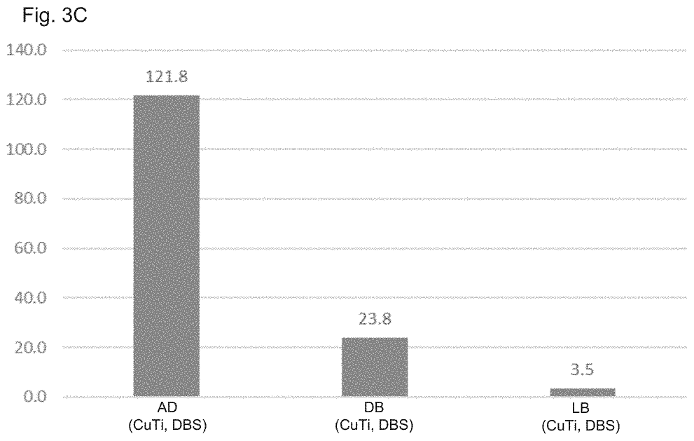

DBS samples were extracted using the three extraction buffers and purified using the CuTi particle process. Purified samples were then assayed with the HIV and the HPV assays and the levels of HIV RNA (Table 10) and cellular DNA (Table 11) were determined from the assay CT values and the standard curves described above herein. The ratio of HIV RNA copies to the amount of cellular DNA (ng) was then used to determine the selectivity of the CuTi purification process for RNA compared to the baseline silica particle purification process (Table 12, FIG. 3B).

TABLE 10
HIV RNA copies per 25 μl sample, comparing CuTi purification
with silica purification (extraction from DBS).

| | Mean | Std | Std Err | Lower | Upper |
|---|---|---|---|---|---|
| Buffer | (#RNA copies) | Dev | Mean | 95% | 95% |

HIV RNA extraction (DBS) with CuTi purification

| | | | | | |
|---|---|---|---|---|---|
| AD buffer | 187.893 | 100.576 | 41.060 | 82.3 | 293.44 |
| DB buffer | 229.173 | 47.601 | 19.433 | 179.2 | 279.13 |
| LB buffer | 121.948 | 11.548 | 4.714 | 109.8 | 134.07 |

HIV RNA extraction (DBS) with silica purification

| | | | | | |
|---|---|---|---|---|---|
| AD buffer | 232.313 | 154.181 | 77.090 | −13.0 | 477.65 |
| DB buffer | 543.699 | 118.199 | 48.254 | 419.7 | 667.74 |
| LB buffer | 231.057 | 112.912 | 50.496 | 90.9 | 371.26 |

TABLE 11
Cellular DNA (ng) per 25 μl sample, comparing CuTi purification
with silica purification (extraction from DBS).

| | Mean | Std | Std Err | Lower | Upper |
|---|---|---|---|---|---|
| Buffer | (ng DNA) | Dev | Mean | 95% | 95% |

Cellular DNA extraction (DBS) with CuTi purification

| | | | | | |
|---|---|---|---|---|---|
| AD buffer | 2.519 | 0.1502 | 0.061 | 2.36 | 2.68 |
| DB buffer | 6.747 | 1.1209 | 0.458 | 5.57 | 7.92 |
| LB buffer | 25.890 | 2.3847 | 0.974 | 23.39 | 28.39 |

Cellular DNA extraction (DBS) with silica purification

| | | | | | |
|---|---|---|---|---|---|
| AD buffer | 378.320 | 28.9800 | 14.490 | 332.21 | 424.43 |
| DB buffer | 395.441 | 81.6324 | 33.326 | 309.77 | 481.11 |
| LB buffer | 181.877 | 38.0202 | 17.003 | 134.67 | 229.09 |

A greater percentage of HIV RNA than of cellular DNA was recovered with CuTi particles (FIG. 3A). Overall recovery of HIV RNA was approximately 40% to 80% of the recovery with the silica process. However, the amount of cellular DNA recovered was much lower using the CuTi particle process with only about 1% to 2% recovered with AD and DB buffers. The ratio of HIV RNA copies per 25 μl to the ng cellular DNA per 25 μl sample increased from 3.5-fold for LB buffer to 23-fold for DB buffer and over 120-fold for AD buffer (Table 12, FIG. 3B and 3C).

TABLE 12
HIVcopies/ngDNA By Extraction from DBS with
CuTi purification and silica purification

| | Mean | | | | |
|---|---|---|---|---|---|
| | (#RNA copies/ | Std | Std Err | Lower | Upper |
| Buffer | ng DNA) | Dev | Mean | 95% | 95% |

HIV RNA/cellular DNA recovery with CuTi purification (DBS)

| | | | | | |
|---|---|---|---|---|---|
| AD buffer | 76.1429 | 45.3716 | 18.523 | 28.53 | 123.76 |
| DB buffer | 34.6904 | 9.6570 | 3.942 | 24.56 | 44.82 |
| LB buffer | 4.7600 | 0.7831 | 0.320 | 3.94 | 5.58 |

HIV RNA/cellular DNA recovery with silica purification (DBS)

| | | | | | |
|---|---|---|---|---|---|
| AD buffer | 0.6252 | 0.40837 | 0.2042 | −0.0246 | 1.275 |
| DB buffer | 1.4571 | 0.55884 | 0.2281 | 0.87 | 2.044 |
| LB buffer | 1.3544 | 0.79778 | 0.3568 | 0.36 | 2.345 |

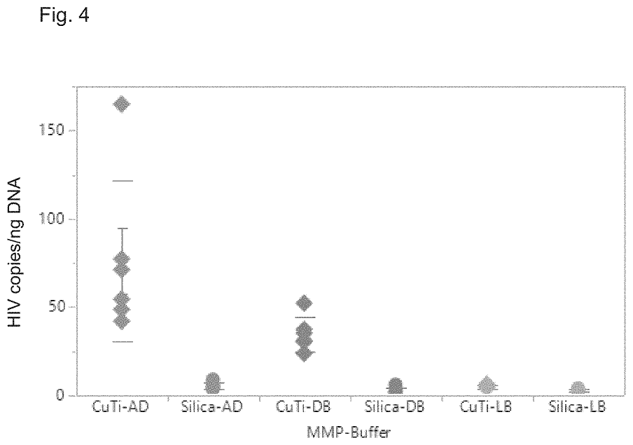

Together, the data presented in Examples 1 and 2 show that extraction from DBS samples is selective for RNA over DNA. The amount of cellular DNA was more reduced in the nucleic acids extracted and purified from DBS samples than was the amount of RNA, as compared to the amount of the respective nucleic acids extracted and purified from whole blood. The data also show that CuTi particle purification preferentially isolated RNA over DNA as compared to silica particle purification. This selectivity was observed with all three extraction buffers. FIG. 4 and Table 13 show increased selectivity of RNA extraction and purification from DBS samples using AD, DB, and LB buffers in combination with CuTi particles versus in combination with silica particles. For DBS samples, the combination of AD buffer extraction and CuTi particle purification (“CuTi-AD”) had the highest overall RNA selectivity, while the combination of DB buffer extraction and CuTi particle purification (“CuTi-DB”) had the second highest overall RNA selectivity.

TABLE 13

| Particle-Buffer | | Std | Std Err | Lower | Upper |
|---|---|---|---|---|---|
| combination | Mean | Dev | Mean | 95% | 95% |
| CuTi-AD | 76.142941 | 45.371628 | 18.522889 | 28.528338 | 123.75754 |
| Silica-AD | 5.1676948 | 1.8568798 | 0.758068 | 3.219019 | 7.1163706 |
| CuTi-DB | 34.69038 | 9.6570178 | 3.942461 | 24.555961 | 44.824799 |
| Silica-DB | 2.7195459 | 1.7110908 | 0.6985499 | 0.9238662 | 4.5152256 |
| CuTi-LB | 4.7599944 | 0.7830997 | 0.3196991 | 3.9381817 | 5.5818071 |
| Silica-LB | 2.6627403 | 0.7124857 | 0.2908711 | 1.9150324 | 3.4104481 |

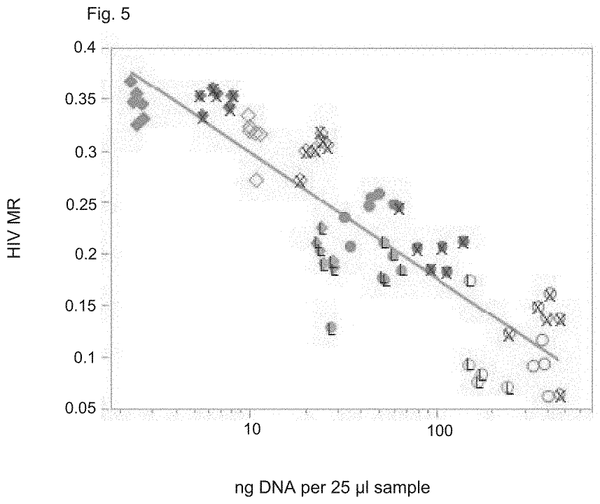

As shown in FIG. 5 and Table 14, Mass Ratio (MR) values were measured for the HIV assays and a bivariate fit analysis was performed. MR values are indicative of the robustness of the assay and can have a bearing on assay sensitivity; reduced amounts of genomic DNA improve MR values. These analyses again illustrated the increased selectivity of RNA extraction and purification for both DBS and whole blood samples using AD or DB buffer in combination with CuTi particle purification. DBS samples extracted and purified using the combination of AD or DB buffer and CuTi particle purification had the highest MR values, followed by whole blood samples extracted and purified using the combination of AD or DB buffer and CuTi particle purification.

TABLE 14
Bivariate fit analysis for mass ratio values
Transformed Fit to Log
HIV MR = 0.4203939 − 0.0528971*Log(ng DNA per 25 μl sample)

| Summary of Fit | | | | |
|---|---|---|---|---|
| RSquare | 0.80214 | | | |
| RSquare Adj | 0.798896 | | | |
| Root Mean | 0.040922 | | | |
| Square Error | | | | |
| Mean of | 0.228476 | | | |
| Response | | | | |
| Observations | 63 | | | |
| (or Sum Wgts) | | | | |
| Analysis of Variance | | Sum of | Mean | |
| Source | DF | Squares | Square | F Ratio |
| Model | 1 | 0.41412853 | 0.414129 | 247.2985 |
| Error | 61 | 0.10215119 | 0.001675 | Prob > F |
| C. Total | 62 | 0.51627971 | | <.0001* |
| Parameter Estimates | | | | |
| Term | Estimate | Std Error | t Ratio | Prob > \|t\| |
| Intercept | 0.4203939 | 0.013248 | 31.73 | <.0001* |
| Log(ng DNA per | −0.052897 | 0.003364 | −15.73 | <.0001* |
| 25 ul sample) | | | | |

### Example 3

### Extraction-Purification Selection: CuTi Extraction Buffers

Three factors influence the degree of RNA selectivity of the method: (1) the extraction step for extracting RNA from DBS samples, as disclosed above herein in Example 1; (2) the purification step for isolating extracted RNA above herein in Example 2; and (3) the extraction buffer used for steps (1) and (2). The extraction buffers (AD, DB, and LB buffers as described above herein) were comprised of three components, guanidine isothiocyanate (GITC), Tween®-20, and a buffer.

### Results and Discussion

### (1) Extraction Buffer Composition and Overall Performance With DBS Samples

The AD, DB, and LB buffers were tested with DBS samples, with both the CuTi particle process and the silica particle process as described in Example 2. DBS extraction used full strength buffers, and extracts were diluted to approximately 1.8 M GITC for CuTi particle purification. Diluted Tween®-20 concentrations were 4.25%, 2.5%, and 4% for the AD, DB, and LB buffers respectively.

### (2) GITC Level in Extraction

DBS samples were extracted as above in 1.3 ml of buffer at 55° C. for 30 minutes. The extracts were diluted in CuTi purification to 1.75 M GITC as indicated in Table 15. The lysis-capture stage of the purification was at 50° C. for 20 minutes after the addition of 40 μl of the CuTi particles. Samples were processed and assayed as described above.

TABLE 15

| | | | Buffer | | ml water |
|---|---|---|---|---|---|
| Buffer | GITC M | Tween ®-20 | concentration | pH | in dilution |
| 3.2M-5.45 | 3.2 | 7.5% | 50 mM | 5.45 | 0.83 |
| 3.2M-5.6 | 3.2 | 7.5% | 50 mM | 5.6 | 0.83 |
| 3.5M-5.45 | 3.5 | 7.5% | 50 mM | 5.45 | 1 |
| 3.5M-5.6 | 3.5 | 7.5% | 50 mM | 5.6 | 1 |
| 3.5M-5.7 | 3.5 | 7.5% | 50 mM | 5.7 | 1 |
| 4.0M-5.6 | 4 | 7.5% | 50 mM | 5.6 | 1.29 |
| 4.5M-5.6 | 4.5 | 7.5% | 50 mM | 5.6 | 1.57 |

As summarized in Tables 16-18, the CuTi particle method had a higher RNA selectivity with a lower GITC concentration in the extraction buffer. The extraction buffers had similar pH, buffer concentrations, and Tween®-20 levels. All buffers were diluted to 1.75M GITC in the purification step of the procedure. HIV RNA recovery was similar between the different buffers but the amount of cellular DNA increased in the higher GITC concentration extractions (Table 17).

TABLE 16
HIV copies/25 μl sample by GITC concentration

| Level | Mean | Std | Std Err | Lower | Upper |
|---|---|---|---|---|---|
| (GITC M) | (# RNA copies) | Dev | Mean | 95% | 95% |
| 3.2M-5.45 | 27.2783 | 16.6290 | 8.3145 | 0.818 | 53.739 |
| 3.2M-5.6 | 43.5943 | 19.9425 | 8.9185 | 18.832 | 68.356 |
| 3.5M-5.45 | 20.8876 | 5.9816 | 2.4420 | 14.610 | 27.165 |
| 3.5M-5.6 | 37.2629 | 19.2727 | 5.8109 | 24.315 | 50.210 |
| 3.5M-5.7 | 24.5409 | 6.5968 | 3.2984 | 14.044 | 35.038 |
| 4.0M-5.6 | 32.2543 | 13.0955 | 5.3462 | 18.511 | 45.997 |
| 4.5M-5.6 | 34.9777 | 13.4121 | 6.7060 | 13.636 | 56.319 |

TABLE 17
Cellular DNA (ng)/25 μl sample by GITC concentration

| Level | Mean | Std | Std Err | Lower | Upper |
|---|---|---|---|---|---|
| (GITC M) | (ng DNA) | Dev | Mean | 95% | 95% |
| 3.2M-5.45 | 1.14017 | 0.090697 | 0.03703 | 1.0450 | 1.2353 |
| 3.2M-5.6 | 1.33799 | 0.202167 | 0.08253 | 1.1258 | 1.5501 |
| 3.5M-5.45 | 1.39560 | 0.073862 | 0.03015 | 1.3181 | 1.4731 |
| 3.5M-5.6 | 1.39008 | 0.203728 | 0.05881 | 1.2606 | 1.5195 |
| 3.5M-5.7 | 1.41564 | 0.099316 | 0.04055 | 1.3114 | 1.5199 |
| 4.0M-5.6 | 2.01407 | 0.276438 | 0.11286 | 1.7240 | 2.3042 |
| 4.5M-5.6 | 2.80250 | 0.629132 | 0.25684 | 2.1423 | 3.4627 |

TABLE 18
HIV RNA/ng cellular DNA per 25
μl sample by GITC concentration

| | Mean | | | | |
|---|---|---|---|---|---|
| Level | (RNA copies/ | Std | Std Err | Lower | Upper |
| (GITC M) | ng DNA) | Dev | Mean | 95% | 95% |
| 3.2M-5.45 | 23.2772 | 13.5072 | 6.7536 | 1.784 | 44.770 |
| 3.2M-5.6 | 32.7017 | 13.0142 | 5.8201 | 16.542 | 48.861 |
| 3.5M-5.45 | 15.0144 | 4.3889 | 1.7918 | 10.409 | 19.620 |
| 3.5M-5.6 | 28.5361 | 17.4911 | 5.2738 | 16.785 | 40.287 |
| 3.5M-5.7 | 16.8314 | 4.1703 | 2.0851 | 10.196 | 23.467 |
| 4.0M-5.6 | 16.3491 | 7.2061 | 2.9419 | 8.787 | 23.911 |
| 4.5M-5.6 | 13.1245 | 7.2649 | 3.6324 | 1.564 | 24.685 |

### (3) GITC Level in Purification: (All Extracted With DB Buffer Then Diluted in Purification).

DBS samples were extracted as described above in 1.3 ml of DB buffer at 55° C. for 30 minutes. The extracts were diluted in the purification as indicated below (Table 19). The lysis-capture stage of the purification was at 50° C. for 20 minutes after the addition of 40 μl of CuTi particles. The samples were processed and assayed as described above.

TABLE 19

| | | ml water in | Purification |
|---|---|---|---|
| DB GITC M | ml extract | dilution | GITC M |
| 3.5 | 1 | 1.75 | 1.27 |
| 3.5 | 1 | 1.5 | 1.40 |
| 3.5 | 1 | 1.25 | 1.56 |
| 3.5 | 1 | 1 | 1.75 |
| 3.5 | 1 | 0.75 | 2.00 |
| 3.5 | 1 | 0.5 | 2.33 |
| 3.5 | 1 | 0 | 3.50 |

As shown in Tables 20-22, the CuTi particle method had a higher RNA selectivity with a lower GITC concentration in the purification. HIV RNA recovery was highest at the 1.75 M GITC and 2 M GITC dilutions. Those purification concentrations showed the highest RNA selectivities. The amount of cellular DNA increased in the 2.33 and 3.5 M GITC concentration purifications.

TABLE 20
HIV copies/25 μl sample by GITC concentration

| Level | Mean | Std | Std Err | Lower | Upper |
|---|---|---|---|---|---|
| ([GITC]) | (# RNA copies) | Dev | Mean | 95% | 95% |
| 3.2M-5.45 | 73.699 | 37.5938 | 16.812 | 27.02 | 120.38 |
| 3.2M-5.6 | 126.637 | 44.6871 | 18.243 | 79.74 | 173.53 |
| 3.5M-5.45 | 129.545 | 64.4519 | 26.312 | 61.91 | 197.18 |
| 3.5M-5.6 | 156.515 | 59.5862 | 17.201 | 118.66 | 194.37 |
| 3.5M-5.7 | 153.101 | 65.7975 | 26.862 | 84.05 | 222.15 |
| 4.0M-5.6 | 119.520 | 44.4669 | 18.154 | 72.85 | 166.18 |
| 4.5M-5.6 | 130.659 | 29.5893 | 12.080 | 99.61 | 161.71 |

TABLE 21
Cellular DNA (ng)/25 μl sample by GITC concentration

| Level | Mean | Std | Std Err | Lower | Upper |
|---|---|---|---|---|---|
| ([GITC]) | (ng DNA) | Dev | Mean | 95% | 95% |
| 3.2M-5.45 | 1.25662 | 0.118554 | 0.04840 | 1.1322 | 1.3810 |
| 3.2M-5.6 | 1.16139 | 0.189046 | 0.07718 | 0.9630 | 1.3598 |
| 3.5M-5.45 | 1.05510 | 0.312051 | 0.12739 | 0.7276 | 1.3826 |
| 3.5M-5.6 | 1.40200 | 0.280834 | 0.08107 | 1.2236 | 1.5804 |
| 3.5M-5.7 | 1.26708 | 0.416906 | 0.17020 | 0.8296 | 1.7046 |
| 4.0M-5.6 | 1.97074 | 0.333466 | 0.13614 | 1.6208 | 2.3207 |
| 4.5M-5.6 | 3.03111 | 0.571917 | 0.23348 | 2.4309 | 3.6313 |

TABLE 22
HIV RNA/ng cellular DNA per 25
μl sample by GITC concentration

| | Mean | | | | |
|---|---|---|---|---|---|
| Level | (RNA copies/ | Std | Std Err | Lower | Upper |
| ([GITC]) | ng DNA) | Dev | Mean | 95% | 95% |
| 3.2M-5.45 | 57.580 | 29.2272 | 13.071 | 21.290 | 93.87 |
| 3.2M-5.6 | 110.052 | 38.1868 | 15.590 | 69.977 | 150.13 |
| 3.5M-5.45 | 117.090 | 47.0731 | 19.217 | 67.690 | 166.49 |
| 3.5M-5.6 | 115.089 | 45.2388 | 13.059 | 86.345 | 143.83 |
| 3.5M-5.7 | 126.158 | 46.8357 | 19.121 | 77.006 | 175.31 |
| 4.0M-5.6 | 59.696 | 16.8683 | 6.886 | 41.993 | 77.40 |
| 4.5M-5.6 | 43.409 | 8.8591 | 3.617 | 34.112 | 52.71 |

### (4) RNA Selectivity by Tween® Concentration in DBS Buffer

Extraction buffers were made with various levels of Tween®-20 and used to test the extraction-purification process with the CuTi particles. Extractions were done with 1.3 ml of buffer as described above in this Example and 1 ml of the extracted samples were purified with a 1 ml dilution with water and the addition of 40 μl of CuTi particles. The purified targets were assayed as described above in this Example.

As summarized in Tables 23-24, the CuTi particle method had a higher RNA and DNA recovery with a lower Tween®-20 concentration in the extraction-purifications. Both RNA and DNA were recovered at lower levels with higher levels of Tween®-20. RNA selectivity did not appear to be influenced greatly by the Tween®-20 concentration at the values tested (Table 25).

TABLE 23
HIV copies/25 μl sample by Tween ®-20 percentage

| Level (% | Mean | Std | Std Err | Lower | Upper |
|---|---|---|---|---|---|
| Tween ®) | (# RNA copies) | Dev | Mean | 95% | 95% |
| 5% | 42.4573 | 20.6757 | 8.4408 | 20.759 | 64.155 |
| 7.5% | 42.6999 | 22.3827 | 9.1377 | 19.211 | 66.189 |
| 10% | 27.4145 | 10.0942 | 4.1209 | 16.821 | 38.008 |
| 13% | 19.6744 | 6.4347 | 2.8777 | 11.685 | 27.664 |

TABLE 24
Cellular DNA (ng)/25 μl sample by Tween ®-20 percentage

| Level (% | Mean | Std | Std Err | Lower | Upper |
|---|---|---|---|---|---|
| Tween ®) | (ng DNA) | Dev | Mean | 95% | 95% |
| 5% | 1.44222 | 0.174181 | 0.07111 | 1.2594 | 1.6250 |
| 7.5% | 1.32832 | 0.159525 | 0.06513 | 1.1609 | 1.4957 |
| 10% | 0.90000 | 0.100057 | 0.04085 | 0.7950 | 1.0050 |
| 13% | 0.49745 | 0.092784 | 0.03788 | 0.4001 | 0.5948 |

TABLE 25
HIV RNA/ng cellular DNA per 25 μl sample
by Tween ®-20 percentage

| | Mean | | | | |
|---|---|---|---|---|---|
| Level (% | (RNA copies/ | Std | Std Err | Lower | Upper |
| Tween ®) | ng DNA) | Dev | Mean | 95% | 95% |
| 5% | 29.5327 | 13.9248 | 5.6848 | 14.920 | 44.146 |
| 7.5% | 32.0878 | 15.5164 | 6.3345 | 15.804 | 48.371 |
| 10% | 30.0151 | 9.0396 | 3.6904 | 20.529 | 39.502 |
| 13% | 41.7589 | 21.5521 | 9.6384 | 14.998 | 68.519 |

### (5) RNA Selectivity in Extraction-Purification by pH

DB buffer pH was adjusted to various levels by the addition of either dilute acetic acid or sodium hydroxide. Extractions were done with 1.3 ml of buffer as described above in this Example and 1 ml of the extracted samples were purified with a 1 ml dilution with water and the addition of 40 μl of CuTi particles. Purified targets were assayed as described above herein.

As summarized in Tables 26-28, the CuTi particle method had higher RNA selectivity with a lower pH extraction buffer. Extracts were all made the DB buffer and diluted to different pH levels in the purification step of the procedure. HIV RNA recovery was consistent across the pH range tested but DNA levels were lower with pH 6.24 and below. RNA selectivity was also greatest at levels of pH 6.24 and below.

TABLE 26
HIV copies/25 μl sample by pH

| Level | Mean | Std | Std Err | Lower | Upper |
|---|---|---|---|---|---|
| (pH) | (# RNA copies) | Dev | Mean | 95% | 95% |
| pH 5.3 | 50.9234 | 19.2505 | 7.859 | 30.721 | 71.126 |
| pH 5.5 | 37.0155 | 6.5217 | 3.261 | 26.638 | 47.393 |
| pH 5.6 | 52.8911 | 20.6821 | 10.341 | 19.981 | 85.801 |
| pH 5.9 | 40.0526 | 22.8698 | 6.602 | 25.522 | 54.583 |
| pH 6.24 | 38.9173 | 15.8696 | 6.479 | 22.263 | 55.571 |
| pH 6.58 | 38.0486 | 16.6586 | 6.801 | 20.567 | 55.531 |
| pH 6.96 | 49.9181 | 22.3112 | 9.109 | 26.504 | 73.332 |

TABLE 27
Cellular DNA (ng)/25 μl sample by pH

| Level | Mean | Std | Std Err | Lower | Upper |
|---|---|---|---|---|---|
| (pH) | (ng DNA) | Dev | Mean | 95% | 95% |
| pH 5.3 | 1.91168 | 0.510373 | 0.20836 | 1.3761 | 2.4473 |
| pH 5.5 | 2.18511 | 0.543580 | 0.27179 | 1.3202 | 3.0501 |
| pH 5.6 | 1.70589 | 0.176499 | 0.08825 | 1.4250 | 1.9867 |
| pH 5.9 | 1.90391 | 0.247355 | 0.07141 | 1.7468 | 2.0611 |
| pH 6.24 | 2.31136 | 0.819094 | 0.33439 | 1.4518 | 3.1709 |
| pH 6.58 | 3.48326 | 0.986432 | 0.40271 | 2.4481 | 4.5185 |
| pH 6.96 | 4.42724 | 0.519860 | 0.21223 | 3.8817 | 4.9728 |

TABLE 28
HIV RNA/ng cellular DNA per 25
μl sample by GITC concentration

| | Mean | | | | |
|---|---|---|---|---|---|
| Level | (RNA copies/ | Std | Std Err | Lower | Upper |
| (pH) | ng DNA) | Dev | Mean | 95% | 95% |
| pH 5.3 | 28.7261 | 13.5631 | 5.5371 | 14.492 | 42.960 |
| pH 5.5 | 18.0426 | 6.0300 | 3.0150 | 8.447 | 27.638 |
| pH 5.6 | 30.5590 | 10.9002 | 5.4501 | 13.214 | 47.904 |
| pH 5.9 | 20.8597 | 11.6268 | 3.3564 | 13.472 | 28.247 |
| pH 6.24 | 20.7557 | 14.3531 | 5.8596 | 5.693 | 35.818 |
| pH 6.58 | 11.5241 | 5.7536 | 2.3489 | 5.486 | 17.562 |
| pH 6.96 | 11.5833 | 5.8472 | 2.3871 | 5.447 | 17.720 |

### (6) RNA Selectivity With Elution Temperature

DBS samples were extracted as above in 1.3 ml of DB buffer at 55° C. for 30 minutes, and extracts were diluted for purification as indicated above in this Example. The lysis-capture stage of the purification step was performed at 50° C. for 20 minutes after the addition of 40 μl of CuTi particles. Samples were processed as described above except that samples were eluted at 60° C., 65° C., 70° C., and 75° C. instead of only at 75° C. The eluates were assayed as described above herein.

As summarized in Tables 29-31, CuTi particle purification had a higher RNA selectivity with a higher elution temperature. Similar levels of DNA were recovered across the temperature range tested (Table 30) but the amount of RNA detected increased with the higher elution temperatures (Table 39).

TABLE 29
HIV copies/25 μl sample by elution temperature

| Level | Mean | Std | Std Err | Lower | Upper |
|---|---|---|---|---|---|
| (° C.) | (# RNA copies) | Dev | Mean | 95% | 95% |
| 60° C. | 62.144 | 35.3678 | 14.439 | 25.028 | 99.26 |
| 65° C. | 95.169 | 20.5870 | 8.405 | 73.564 | 116.77 |
| 70° C. | 113.011 | 24.8567 | 10.148 | 86.926 | 139.10 |
| 75° C. | 146.385 | 73.3266 | 29.935 | 69.434 | 223.34 |

TABLE 30
Cellular DNA (ng)/25 μl sample by elution temperature

| Level | Mean | Std | Std Err | Lower | Upper |
|---|---|---|---|---|---|
| (° C.) | (ng DNA) | Dev | Mean | 95% | 95% |
| 60° C. | 1.89676 | 0.155914 | 0.06365 | 1.7331 | 2.0604 |
| 65° C. | 1.75307 | 0.644855 | 0.26326 | 1.0763 | 2.4298 |
| 70° C. | 1.95013 | 0.130324 | 0.05320 | 1.8134 | 2.0869 |
| 75° C. | 1.88420 | 0.128254 | 0.05236 | 1.7496 | 2.0188 |

TABLE 31
HIV RNA/ng cellular DNA per 25
μl sample by elution temperature

| | Mean | | | | |
|---|---|---|---|---|---|
| Level | (RNA copies/ | Std | Std Err | Lower | Upper |
| (° C.) | ng DNA) | Dev | Mean | 95% | 95% |
| 60° C. | 32.7058 | 18.0757 | 7.379 | 13.737 | 51.68 |
| 65° C. | 63.6301 | 30.8934 | 12.612 | 31.209 | 96.05 |
| 70° C. | 58.6466 | 15.4082 | 6.290 | 42.477 | 74.82 |
| 75° C. | 77.3093 | 36.4917 | 14.898 | 39.014 | 115.60 |

### (7) Extraction Buffer RNA Selectivity Summary

The experiments described above herein illustrate preferential RNA selection at both the extraction of the sample from the DBS paper disc and at the purification of the nucleic acids from the sample extracts. The level of GITC in the buffer has been shown to be an important factor at both the extraction and purification stages. The pH of the buffer is also an important factor in the two-stage method as shown above. The Tween®-20 concentration may not influence the RNA selectivity but is important in the overall recovery of nucleic acids. The elution temperature also has an influence on the preferential recovery of RNA over cellular DNA.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of extracting RNA molecules from a sample dried on a solid carrier, the method comprising:
(a) providing a liquid biological sample dried on a solid carrier, wherein the liquid biological sample comprises nucleic acids including RNA molecules;
(b) providing an extraction buffer comprising less than 3.5 M GITC;
(c) contacting the solid carrier with the extraction buffer, thereby releasing RNA molecules from the solid carrier into the extraction buffer;
(d) isolating the extraction buffer of step (c) containing released RNA molecules;
(e) suspending a plurality of copper-titanium oxide-coated (CuTi) magnetic particles in the isolated extraction buffer and incubating under conditions appropriate for binding of the released RNA molecules by the plurality of suspended CuTi particles;
(f) capturing the plurality of CuTi particles and bound RNA molecules by application of a magnetic field;
(g) removing the extraction buffer; and
(h) contacting the plurality of CuTi particles and bound RNA molecules with an elution buffer, under conditions appropriate for release of the bound RNA molecules into the elution buffer.

2. The method of claim 1, wherein the liquid biological sample is whole blood.

3. The method of claim 1, wherein the liquid biological sample dried on a solid carrier is a dried blood spot (DBS).

4. The method of claim 1, wherein the liquid biological sample dried on a solid carrier is suspected of containing a virus.

5. The method of claim 4, wherein the virus is human immunodeficiency virus 1 (HIV-1).

6. The two-step method of claim 4, wherein the virus is human papilloma virus (HPV).

7. The method of claim 1, wherein the extraction buffer further comprises greater than 5% Tween®-20 and has a pH less than 6.0.

8. The method of claim 1, wherein the extraction buffer comprises 3.2 M GITC, 7.5% Tween®-20, and has a pH of 5.6.

9. The method of claim 1, wherein the extraction buffer comprises less than 3.2 M GITC, 7.5% Tween®-20, and has a pH less than 6.0.

10. The method of claim 1, wherein step (e) further comprises drawing the sequestered plurality of CuTi particles through an aqueous gel by means of a magnetic force, and step (g) is not performed.

11. The method of claim 1, further wherein the sequestered plurality of CuTi particles are drawn through the aqueous gel directly into the elution buffer of step (h).

12. The method of claim 1, wherein the elution buffer comprises a low ionic strength buffer.

13. The two-step method of claim 1, wherein the elution buffer is water.

14. The method of claim 1, wherein the plurality of CuTi particles is present in a molar excess relative to the plurality of RNA molecules in the sample.

15. The method of claim 1, wherein the method is automated.

16. The method of claim 1, wherein the solid carrier comprises filter paper.

17. The method of claim 1, wherein the method further comprises (i) diagnosing a viral infection in a subject, wherein the diagnosing comprises:
(1) obtaining a nucleotide sequence of the released RNA molecules, or of a template-directed polymerization product thereof; and
(2) comparing the obtained nucleotide sequence of the released RNA molecules, or the template-directed polymerization product thereof, with a specific nucleotide sequence known to be present in virally infected cells, wherein a match between the compared nucleotide sequences is diagnostic of the viral infection in the subject.

18. The method of claim 17, wherein the viral infection is an HIV infection.
